(19) United States
(12) Patent Application Publication
(10) Pub. No.: US 2004/0210254 A1
(43) Pub. Date: Oct. 21, 2004

(54) Method and apparatus for electromagnetic stimulation of nerve, muscle, and body tissues

(76) Inventors: Daniel R. Burnett, Menlo Park, CA (US); Shane Mangrum, Salt Lake City, UT (US); Francesco Lisi, Genova (IT)

Correspondence Address: MAINE & ASMUS, 100 MAIN STREET, P O BOX 3445, NASHUA, NH, 03061-3445, US

(21) Appl. No.: 10/839,364

(22) Filed: May 5, 2004

Related U.S. Application Data
(63) Continuation-in-part of application No. PCT/US03/03028, filed on Feb. 3, 2003.
Application No. PCT/US03/03028 is a continuation of application No. 10/266,535, filed on Oct. 8, 2002, now abandoned.
Application No. PCT/US03/03028 is a continuation-in-part of application No. 10/077,434, filed on Feb. 19, 2002, now Pat. No. 6,701,185.
(60) Provisional application No. 60/467,693, filed on May 5, 2003.
(60) Provisional application No. 60/380,132, filed on May 6, 2002.

(51) Int. Cl. A61N 1/00

(52) U.S. Cl. 607/2

(57) ABSTRACT

A system is disclosed for pulsed electromagnetic stimulation of a target member. The system comprises a pulse control and generation unit, a conformable member appliance coupled to the pulse control and generation unit, and an insulated conductive material disposed within the appliance, the insulated conductive material is disposed proximate to the target member and produces a pulsed magnetic field when an electrical pulse is passed through the conductive material by the pulse control and generation unit.

### RELATED APPLICATIONS

This application claims the benefit of U.S. Provisional Application No. 60/467,693, filed May 5, 2003. In addition, this application is a continuation-in-part of P.C. T. Application No. PCT/US03/03028, designating the United States, filed Feb. 3, 2003, which in turn claims priority to U.S. application Ser. No. 10/266,535 filed Oct. 8, 2002, which claims benefit of U.S. Provisional Application No. 60/380,132, filed May 6, 2002, and is also a continuation in part of, is related to, and claims priority of co-pending United States Non-Provisional application Ser. No. 10/077,434, filed Feb. 19, 2002. Each of these applications is herein incorporated in its entirety by reference.

### FIELD OF THE INVENTION

The present invention relates to the field of medical devices, in particular electromagnetic stimulating devices for stimulation of nerve, muscle, and/or other body tissues with applications in the field of medicine.

### BACKGROUND OF THE INVENTION

The concept of pulsed electromagnetic stimulation (PES) was first observed by the renowned scientist Michael Faraday in 1831. Faraday was able to demonstrate that time varying, or pulsed electromagnetic fields have the potential to induce current in a conductive object. Faraday's experimental setup was simple. He found that by passing strong electric current through a coil of wire he was able to produce pulsed electromagnetic stimuli. This pulsed electromagnetic stimulus was able to induce the flow of current in a nearby electrically conductive body.

In the years since the discoveries of Faraday, pulsed electromagnetic stimulators have found application in countless areas of scientific investigation. In 1965, the scientists Bickford and Freming demonstrated the use of electromagnetic stimulation to induce conduction within nerves of the face. Later, in 1982 Polson et al., U.S. Pat. No. 5,766,124 produced a device capable of stimulating peripheral nerves of the body. This device was able to stimulate peripheral nerves of the body sufficiently to cause muscle activity, recording the first evoked potentials from electromagnetic stimulation. One of the earliest practical applications of electromagnetic stimulating technology took the form of a bone growth stimulator a device that employed low frequency pulsed electromagnetic fields (PEMF) to stimulate bone repair. They first found use approximately 20 years ago in the treatment of non-healing fractures, and are slowly becoming the standard of care for this condition.

As investigators have studied the effects of electromagnetic fields on fracture healing, it has been demonstrated that PEMFs can not only facilitate fracture healing but also promote numerous other positive effects on the human body, including: (1) causing muscles to contract, (2) altering nerve signal transmission to decrease experienced pain, and (3) causing new cell growth in cartilage. These powerful effects of pulsed electromagnetic stimulation have been well established in laboratory studies of animal models and also in multiple large, double blind, placebo-controlled studies of human subjects published in the medical literature.

Existing pulsed electromagnetic stimulation devices have taken a number of different forms in attempts to treat various medical conditions. These different forms have resulted in two broad categories of coil arrangements for the generation of PEMFs: (1) planar or semi-planar designs with tightly wound coils, and (2) solenoid coils. Flat, wound coils create electromagnetic fields that degrade rapidly over a short distance as they pulse away from the inducing coil.

Solenoid type coils create pulsed electromagnetic fields inside the coil that are relatively uniform throughout, with peak field strength at the center of the coil. Examples of existing devices with tightly wound coil arrangements include:

Erickson's U.S. Pat. No. 5,181,902, Jan. 26, 1993, which describes a device using a double transducer system with, contoured, flat wound transducers intended to generate therapeutic flux-aided electromagnetic fields in the body. The device is suggested to be conformed to the contour of the patient's back and incorporates an adjustable belt into the design. This system, as it is described, is disadvantageous in at least two respects. First, the flat, wound nature of the coil in this device is limited in its delivery of pulsed electromagnetic fields to deep tissues of the body. Second, the rigid nature of this device, intended to provide bracing for patients recovering from spinal fusion surgeries, may prove uncomfortable to some patients, especially in delivering therapy to regions of the body other than the back, such as the knee, elbow, hand, or other joints and tissues.

. U.S. Pat. No. 6,086,525, which discloses a device that has a single coil in the shape of a “C” where the intensity of the electromagnetic field is between the ends of the “C”. That point must be employed directly over the target nerve or muscle to be stimulated. The coil is toroidal in configuration and utilizes a unique core of vanadium permendur in the preferred form. One of the disadvantages of this device is that it requires a trained technician to treat the patient and to properly hand hold the open end of the “C” over the targeted nerve or muscle to be stimulated. The device is not portable and is designed for use in hospitals or similar institutions. Also the vanadium permendur core is required to increase the strength of the electromagnetic field to be strong enough to be effectively used. The design, shape and configuration described in Davey and other prior art devices, require the electromagnetic stimulator to be hand operated during use.

Tepper in U.S. Pat. No. 5,314,401, May 24, 1994 describes a pulsed electromagnetic field transducer that is intended to be conformable to the contour of a patient's body. The PEMF transducer in this application as having a desired form and sufficient rigidity to maintain an anatomical contour. This system is disadvantageous in a number of respects. First, the desired contouring of this device will require that a significant number of different sizes be manufactured to accommodate the contours of an endless variety of body shapes. Second, the intended device does not incorporate markings to ensure that the device is placed in a correct alignment over the targeted area of the body. Finally, this proposed device utilizes flat, wound coils, providing PEMFs that do not penetrate as deeply or as uniformly into body tissues as those fields produced by solenoid coils.

In U.S. Pat. No. 6,179,770 B1, Jan. 30, 2001, Mould describes dual coil assemblies in a magnetic stimulator for neuro-muscular tissue, with cooling provided for the transducer coil. This device is intended to be held by a trained user over the targeted regions of the body in order to deliver PEMF therapy. The design of this device is limited by the difficult nature of manipulating a single coil and the cost-intensive requirement of using highly skilled medical personnel for operation.

Parker in U.S. Pat. No. 6,155,966, Dec. 5, 2000 describes a wearable article with a permanent magnet/electromagnet combination device to be used for toning tissue with focused, coherent EMF. This device is disadvantageous in several respects. First, this device is intended to be a hand-held application, with the user applying the device to targeted areas of the body. The hand-held nature of this application creates an inherently inconsistent and non-uniform method for delivery, especially difficult with the intention of the device to provide a focused electromagnetic stimulus. Second, the device combines a static magnet with the electromagnet assembly in an attempt to create a unipolar, negative polarity field. This form of electromagnetic field stimulation has not been demonstrated to be effective in the treatment of osteoarthritis, musculoskeletal pain, or atrophy treatment—conditions for which the present invention will provide therapy.

March's U.S. Pat. No. 6,200,259 B1, Mar. 13, 2001 describes a device with electromagnetic field coils applied front and back to a patient for treating cardiovascular disease by angiogenesis. An EMF dosage plan contemplates, multiple coil implants and pulse variables including carrier frequency, pulse shape, duty cycle, and total time exposed. This device describes the placement of coils around the regions of tissues in which collateralization of blood flow (or angiogenesis) is desired. The design contemplates applications including the use of coils embedded in a cloth wrap, which could be worn as a garment surrounding the body area of interest. Alternatively, a wrap with embedded coils to be placed around an arm or a leg to deliver the desired field is described. The use of PEMF in this application for the purpose of modulation of angiogenesis shows significant promise. The description of this device, however, does not suggest any extension of the electromagnetic phenomenon in circumstances where PEMF stimulation can provide dramatic opportunities for the treatment of osteoarthritis, and musculoskeletal pains including tendonitis, bursitis, and muscle spasms. Furthermore, this invention does not provide for the use of solenoid-type coils for the delivery of PEMF.

Polson's U.S. Pat. No. 5,766,124, Jun. 16, 1998 describes a magnetic stimulator of neuro-muscular tissue. The primary aim of this invention is devise a reserve capacitor providing more efficiency in the control circuitry. The description of the device, however, describes the stimulating coil in broad, generic terms, and does not contemplate application of the coil in any type of body wrap or other specific method for delivering PEMF to targeted areas of the body. As a result, this device is disadvantageous, in the respect that is does not provide for any method or delivery system to provide consistent, uniform PEMF stimulation.

Schweighofer's U.S. Pat. No. 6,123,658, Sep. 26, 2000 describes a magnetic stimulation device which consists of a stimulation coil, a high-voltage capacitor, and a controllable network part. This device is intended to differentiate itself from low-voltage, low current devices by using a specific high voltage, high current design to deliver PEMF for the purpose of triggering action potentials in deep neuromuscular tissue. This device, however, does not contemplate the incorporation of the stimulation coil into ergonomic body wraps for the purpose of delivering consistent, user-friendly therapy. Instead, the coil is described as having a difficult and expensive to use hand-held configuration.

Lin in U.S. Pat. No. 5,857,957, issued Jan. 12, 1999 teaches the use of functional magnetic stimulation for the purpose of inducing a cough function in a mammalian subject. The description of the device provides for the use of hand-held stimulation coil, intended to be placed over the anterior chest of the subject for the purpose of stimulating nerves to induce a cough. This system is disadvantageous in the requirement of hand-held delivery, which is difficult and inconsistent. The description contemplates use of the device in the induction of cough, and does not contemplate extension of the use of the device into other areas of neuromuscular stimulation.

Tepper in U.S. Pat. No. 6,024,691, issued Feb. 15, 2000 describes a cervical collar with integral transducer for PEMF treatment. The description of this device provides for the use of a single coil transducer, formed into the shape of a cervical collar. This system is disadvantageous in several respects. First, this device does not provide for the use of solenoid-type coils in the delivery of PEMF, which can provide a more uniform and consistent signal. Second, the semi-rigid design of the collar complicates the delivery of PEMF to persons of differing body sizes. That is, for a person with a larger than average (or smaller than average) size neck, the design and semi-rigid nature of the device would make an exact fit difficult, thereby diminishing the effectiveness of any delivered therapy. Furthermore, this device is designed to immobilize the neck and is therefore not applicable to most patients. Whereas, with a flexible, ergonomic delivery system for PEMF stimulation, various sizes of wraps can accommodate nearly any type of body habitus. Lastly, the device must be lowered over the head making application difficult versus the invention found in FIGS. 4 and 6 where the coil can be opened to allow entrance of the body part.

Erickson in U.S. Pat. No. 5,401,233, issued Mar. 28, 1995 describes a neck collar device for the delivery of PEMF therapy. The description of this device provides for the use of semi-rigid transducers, intended to be conformable to a selected anatomical contour. This device in disadvantageous in respects similar to those of Pollack U.S. Pat. No. 5,401,233, in that the device does not provide for the use of solenoid-type coils. Furthermore, this device is intended to provide bracing (as might be necessary for the treatment of fractures or after surgery). As a result, the rigidity of the device necessary to serve the bracing function makes the device less comfortable to wear, especially for a person who would not require bracing (such as in the treatment of arthritis, muscle spasm, or other forms of musculoskeletal pain).

While the discussion of prior art above related primarily to devices employing flat, wound coils in the delivery of PEMF, there are a handful of devices that contemplate the use of solenoid-type coils.

Examples Include:

Kolt in U.S. Pat. No. 5,518,495, issued May 21, 1996 describes a coil wound on a large bobbin that permits the insertion of an arm or a leg into the field of the coil for PEMF type therapy. This device is disadvantageous in several respects. First, the described use of a bobbin, around which the wire for the stimulating coil is wound provides for the treatment of certain areas of the body, but is certainly limited in its ability to deliver therapy to areas of the body such as the hips, shoulder, back, neck, etc. That is, the constraints of human anatomy make it nearly impossible to approximate a metal bobbin, and thus the stimulating coil, to regions of the body such as the ball and socket joints of the hip or shoulder, where the round metal bobbin would strike the torso before it allowed the stimulating coils to adequately blanket with therapy the head of arm or and joint in the hip and shoulder. Similarly, the use of a metal bobbin for the delivery of PEMF stimulation to the back would necessitate a large, cumbersome delivery system (into which the entire body would have to fit) in order to adequately deliver stimulation to targeted areas on the back or torso. An ergonomic body wrap, incorporating a solenoid-type coil would prove much more effective in delivering PEMF stimulation directly to the targeted areas.

Second, the device is described as a rigid bobbin through which the extremity is placed. This format makes application more difficult in that the applicator cannot be worn and therefore does not provide for consistent ideal placement of the extremity to maximize field effects. In fact, most designs of a similar nature are clinic-based devices and, therefore, would not be amenable to home healthcare applications as with the current invention.

Third, the device described magnetic field within the bobbin is intended to have a maximum magnetic flux density in the range of 4.5 to 6 gauss. Studies such as by Trock et al in the Journal of Rheumatology 1994; 21(10): 1903-1911, have shown that PEMF stimulation in the range of 15-25 or more gauss are effective in the treatment of osteoarthritis or other musculoskeletal pain conditions.

Pollack in U.S. Pat. No. 5,014,699, issued May 14, 1991 describes a coil wound around the cast on an appendage for the delivery of PEMF treatment to fractured bone. The described device has shown promise for the treatment of fractured bone, especially nonunion or delayed healing fractures. However, the description of the device does not provide for extension of this application to the treatment of other conditions, such as arthritis, musculoskeletal pain, or atrophy. Moreover, the described device does not provide for the extension of the use of an ergonomic, body contoured wrap in the delivery of PEMF.

The present treatments for arthritis, musculoskeletal pain and muscular atrophy consist mostly of traditional medicine including physical therapy and pharmaceuticals with only small inroads made by advancing technology. One of the technologies that has been making significant progress in this field, with multiple scientific studies to support its efficacy, is pulsed electromagnetic stimulation (PES). To date, however, even this technology supported by the literature is not used extensively. This is due, in large part, to the expense associated with repeated clinic visits and trained healthcare operators required to use existing equipment.

Clearly what is needed is a user friendly, portable, pulsed electromagnetic stimulation device having adequate strength and favorable pulse waveform.

### BRIEF SUMMARY OF THE INVENTION

One embodiment of the present invention provides a system for pulsed electromagnetic stimulation of a target member, that system comprising: a pulse control and generation unit; a conformable member appliance coupled to the pulse control and generation unit; an insulated conductive material disposed within the appliance and proximate to the target member and producing a pulsed magnetic field when an electrical pulse is passed through the conductive material by the pulse control and generation unit.

Another embodiment of the present invention provides such a system wherein the conformable member comprises an inflatable layer.

A further embodiment of the present invention provides such a system wherein the inflatable layer comprises a layer of self-inflatable foam.

Still another embodiment of the present invention provides such a system further comprising a pump coupled to the layer of self-inflating foam such that the self-inflating foam can be deflated for insertion of the target member into the appliance.

A still further embodiment of the present invention provides such a system wherein the pump is selected from the group of pumps consisting of manual and automatic pumps.

Even another embodiment of the present invention provides such a system wherein the conductive material comprises at least one spring tensioned wire.

An even further embodiment of the present invention provides such a system wherein the appliance comprises an expandable sleeve.

Yet another embodiment of the present invention provides such a system wherein the expandable sleeve comprises an expandable, conformable fabric selected from the group of fabrics consisting of man made stretch fabrics, knitted fabrics, elasticized fabrics, latex, and rubber.

A yet further embodiment of the present invention provides such a system wherein the insulated conductive material is arrayed in a modified Helmholtz configuration such that first and second coils have an axis normal to an axis of the appliance, the coils conform to the shape of the target member and have a maximum separation equal to the diameter of the coil.

One embodiment of the present invention provides such a system wherein the insulated conductive material is configured in first and second coils arrayed in a classic Helmholtz configuration.

Another embodiment of the present invention provides such a system wherein the insulated conductive material is substantially solenoidal in configuration, and comprises flexible wires configured for folding without damage.

A further embodiment of the present invention provides such a system wherein the appliance is substantially larger than the radius of the target member and is configured to fold over and fasten.

Still another embodiment of the present invention provides such a system wherein the appliance further comprises a conductive layer connected to ground whereby the controller is triggered to interrupt the electrical pulse in the event of a failure in insulation of the conductive material.

A still further embodiment of the present invention provides such a system wherein the conductive layer is disposed between the insulated conductive material and at least one exterior insulating layer.

Even another embodiment of the present invention provides such a system wherein the electrical pulse has a current of between 20 and 50 amps.

An even further embodiment of the present invention provides such a system wherein the a pulse control and generation unit comprises: a current sensor, monitoring electrical current flow through the insulated conductive material; a control circuit, receiving data from the current sensor; a switch controlled by the control circuit; a diode disposed within the pulse generation and control circuit such that when the switch is open, the electric current flows through the insulated conductive material will decay.

Yet another embodiment of the present invention provides such a system further comprising a resistor disposed in series with the insulated conductive material.

A yet further embodiment of the present invention provides such a system wherein the control circuit comprises: an oscillator, the oscillator controlling the frequency of the electrical pulse; and a voltage comparator where by a reference voltage is compared to a signal produced by the current sensor.

One embodiment of the present invention provides such a system wherein the a pulse control and generation unit further comprises a first timer where by a user can program the system to provide the pulsed electromagnetic field for a desired time period.

Another embodiment of the present invention provides such a system wherein the pulse control and generation unit further comprises a second timer whereby the system may be programmed to provide the pulsed electromagnetic filed for no longer than a prescribed time period. The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a perspective view of a pulsed electromagnetic stimulation device configured in accordance with one embodiment of the present invention.

FIG. 2 is a perspective view of appliance of a pulsed electromagnetic stimulation device configured in accordance with one embodiment of the present invention.

FIG. 3 is a perspective view of an open expanded appliance of a pulsed electromagnetic stimulation device configured in accordance with one embodiment of the present invention.

FIG. 4 is a perspective view of a closed appliance of a pulsed electromagnetic stimulation device configured in accordance with one embodiment of the present invention.

FIG. 5 is a perspective view of an expanded appliance of a pulsed electromagnetic stimulation device illustrating an expanded spring wound coil configured in accordance with one embodiment of the present invention.

FIG. 6 is a perspective view of a contracted appliance of a pulsed electromagnetic stimulation device illustrating a relaxed spring wound coil configured in accordance with one embodiment of the present invention.

FIG. 7A is a perspective view of an expanded appliance of a pulsed electromagnetic stimulation device illustrating a classic Helmholtz orientation of coils configured in accordance with one embodiment of the present invention.

FIG. 7B is an elevation view of an expanded appliance of a pulsed electromagnetic stimulation device illustrating a classic Helmholtz orientation of coils configured in accordance with one embodiment of the present invention.

FIG. 8A is a perspective view of an expanded appliance of a pulsed electromagnetic stimulation device illustrating a modified Helmholtz orientation of coils configured in accordance with one embodiment of the present invention.

FIG. 8B is an elevation view of an expanded appliance of a pulsed electromagnetic stimulation device illustrating a modified Helmholtz orientation of coils configured in accordance with one embodiment of the present invention.

FIG. 9 is a perspective view of appliance of a pulsed electromagnetic stimulation device illustrating a deflated inflatable fitting lining configured in accordance with one embodiment of the present invention.

FIG. 10 is a perspective view of appliance of a pulsed electromagnetic stimulation device illustrating an inflated inflatable fitting lining configured in accordance with one embodiment of the present invention.

FIG. 11 is a perspective view of appliance of a pulsed electromagnetic stimulation device illustrating a deflated inflatable fitting lining having a manual inflation pump attached configured in accordance with one embodiment of the present invention.

FIG. 12 is a perspective view of appliance of a pulsed electromagnetic stimulation device illustrating a pump manual inflation nozzle configured in accordance with one embodiment of the present invention.

FIG. 13 is a photograph of an oscilloscope illustrating the waveform of the signal produced by one embodiment of the present invention.

FIG. 14 is a schematic diagram of a pulse generation and control circuit configure according to one embodiment of the present invention.

FIG. 15 is a schematic diagram of a DC power supply configured according to one embodiment of the present inventions.

FIG. 16 is schematic diagram of a current control circuit configured according to one embodiment of the present invention.

### DETAILED DESCRIPTION OF THE INVENTION

The invention is susceptible to many and various embodiments; those embodiments described below should not be interpreted as restrictive, but rather as merely illustrative of the invention.

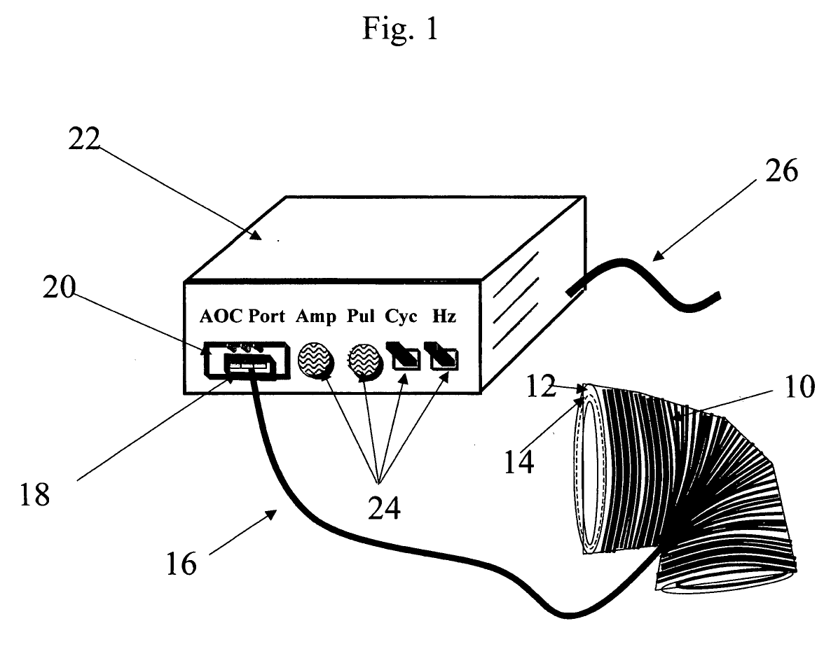

As illustrated in FIG. 1, one embodiment of the present invention provides a Pulsed Electromagnetic Stimulation (PES) appliance 10 coupled to an external logic controller consol 22 providing an electric signal via a connection cable 16. The connection cable 16 mates with a port 20 disposed within the external logic controller consol 22 via a coupler 18. The external logic controller consol also provides a variety of controls 24 whereby the electronic signal may be adjusted by a user as described by a therapist.

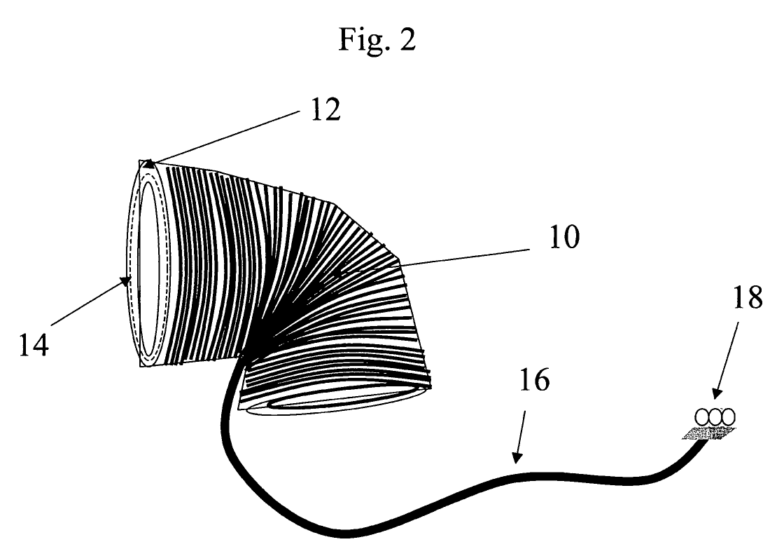

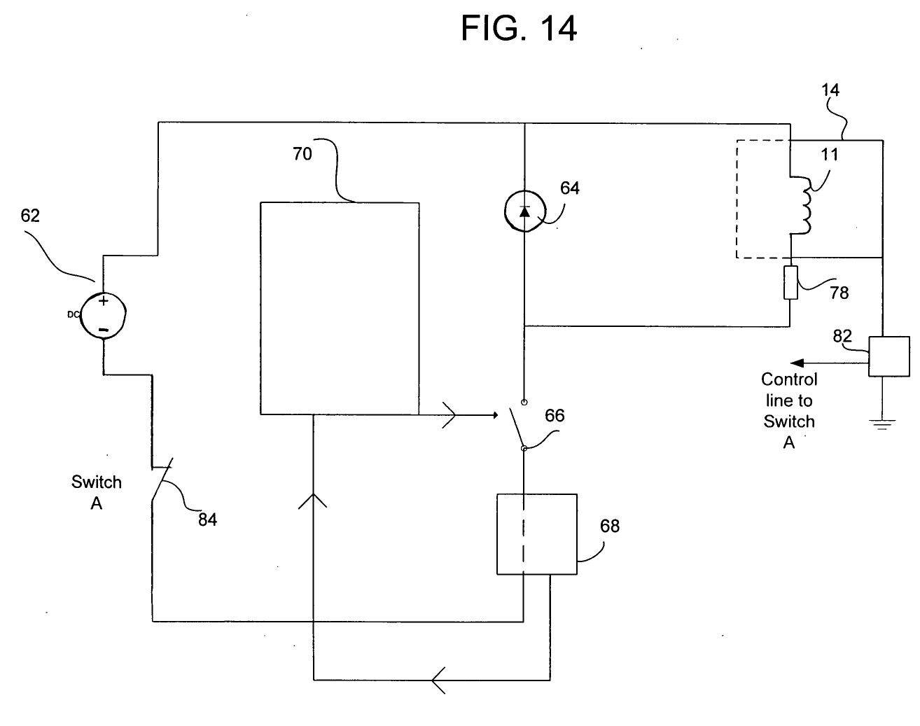

The PES appliance 10 of FIG. 1 is illustrated in greater detail in FIG. 2. The PES appliance 10 comprises at least one coil of wire 11 wrapped in a plurality of twists. This coil of wire 11 may be, as one of ordinary skill in the art will readily appreciate, configured in a variety of configurations within the scope of the present invention. According to one embodiment of the present invention, this coil of wire 11 may be disposed between layers of flexible, conformable, insulating material 12. The coil of wire 11, has, for the sake of clarity been illustrated in solid lines. In some embodiments a grounded conductive layer 14 is disposed within the appliance between the coils 11 and the target member, whereby the controller consol 22 is deactivated in the event of failure of the insulation of the coil 11, as sensed by a voltage sensor 80, illustrated in FIG. 14.

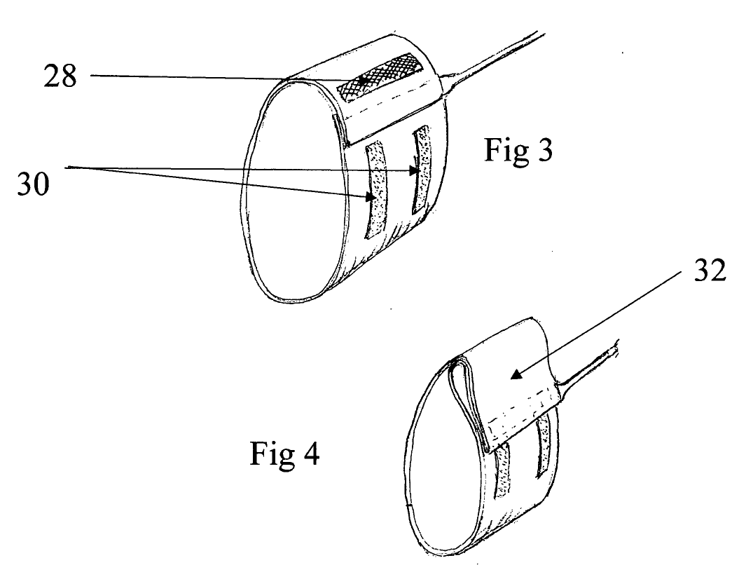

According to one embodiment, illustrated in FIGS. 3 and 4, the wires 11 within the PES Appliance comprise a flexible conductive material. The wires 11 are configured in a substantially solenoidal geometry. The appliance is configured with an adequate circumference to permit a target treatment area to be introduced through the appliance 10. The appliance may then be folded over or gathered. The portion of the appliance 10 that is made redundant by this fold 32 is then secured to provide a snug fit. One skilled in the art will readily appreciate that a variety of means may be used to secure redundant material. Such means may include but are not limited to snaps, hook and eye fasteners, VELCRO® brand fasteners and their equivalents, clips, draw strings, and straps.

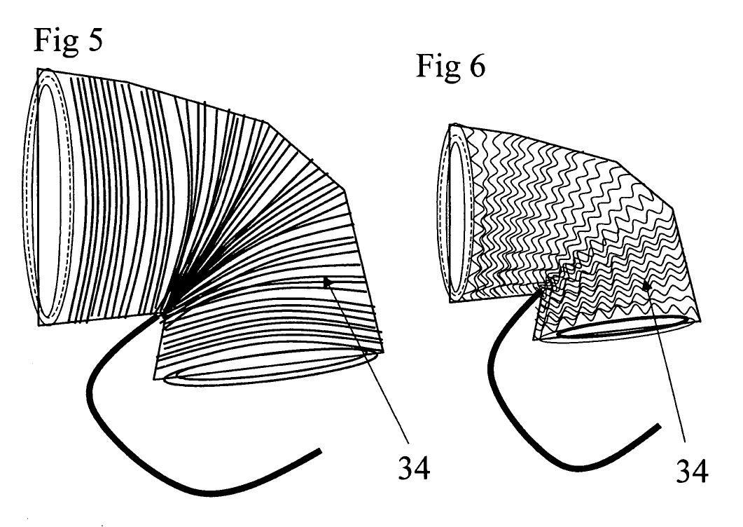

According to one embodiment of the present invention illustrated in FIGS. 5 and 6, the wires 11 are spring-tensioned wire 34. The spring tensioned wires 34 are arrayed in a substantially solenoidal configuration. When such springs are disposed within an expandable fabric, such as LYCRA®, spandex, knit material, elasticized material, rubber, or latex, a patient or therapist may slide the appliance over the target area. The appliance 10 will expand to allow proper placement of the appliance yet once placed, will conform to the target area.

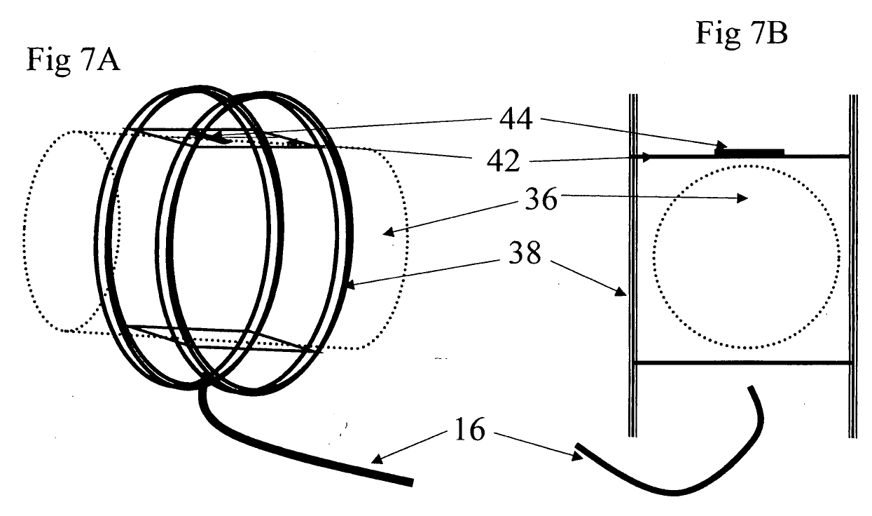

FIGS. 7A and 7B illustrate two views of an embodiment of the present invention utilizing a Helmholtz Coil configuration. This apparatus comprises two parallel coils 38 separated by spacers 42. At least one spacer is configured to open at a joint. This joint is closeable by a latch 44 or other fastener. The patient's knee is disposed between the parallel coils. In this embodiment, both coils are parallel and directly opposite each other separated by a distance equal to the coil radius. The coil may be placed in an ergonomic wrap (not shown) and would utilize moderately flexible spacers 42 to make sure that the proper distances are maintained. The coils will be held at the correct distance by a latching mechanism 44, accessible through the wrap and holding one of the spacers together.

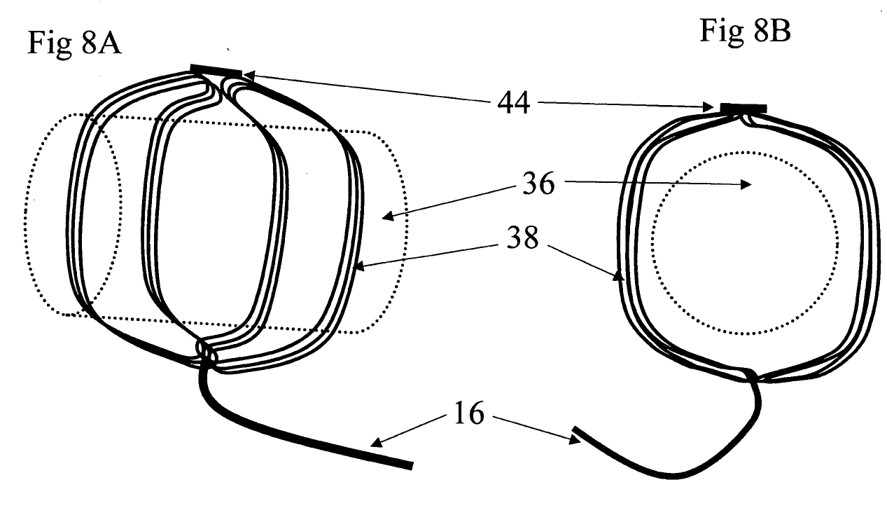

Although similar to that embodiment illustrated in FIGS. 7A and 7B, one embodiment of the present invention, illustrated in FIGS. 8A and 8B provides two conducting coils 38 configured with an ergonomic curve. This curve will allow the coil to be easily placed and latched 21 around the knee 15 while delivering a magnetic field of similar consistency to the more awkward Helmholtz coil of FIGS. 7A and 7B. Once again, these coils will be placed in an ergonomic wrap (not shown) as with all the previous applicators.

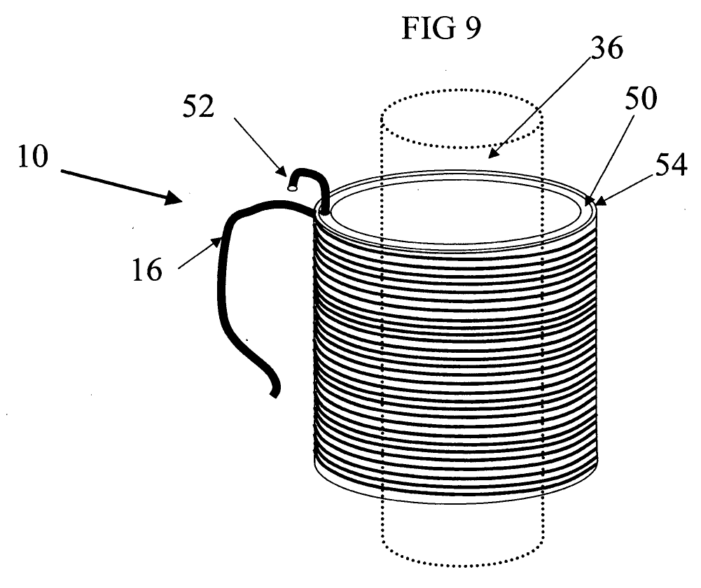

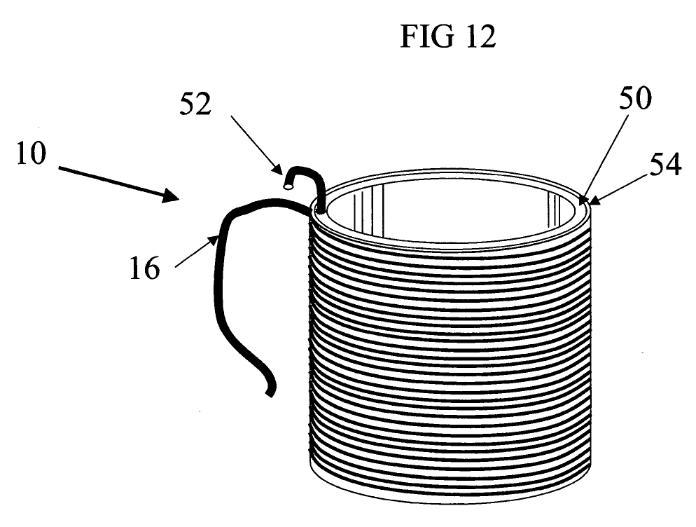

Another embodiment of the present invention, illustrated in FIGS. 9-12 provides a PES appliance 10 having a self-inflating foam layer 50 disposed on the interior of the appliance 10. At least one layer of insulation 54 is provided to protect a user from shock or other injury. At least one air valve 53 is coupled to said self inflating foam layer 50, whereby air is supplied to, or withdrawn from the self inflating foam layer 50, thereby allowing the layer 53 to inflate or deflating the layer 53.

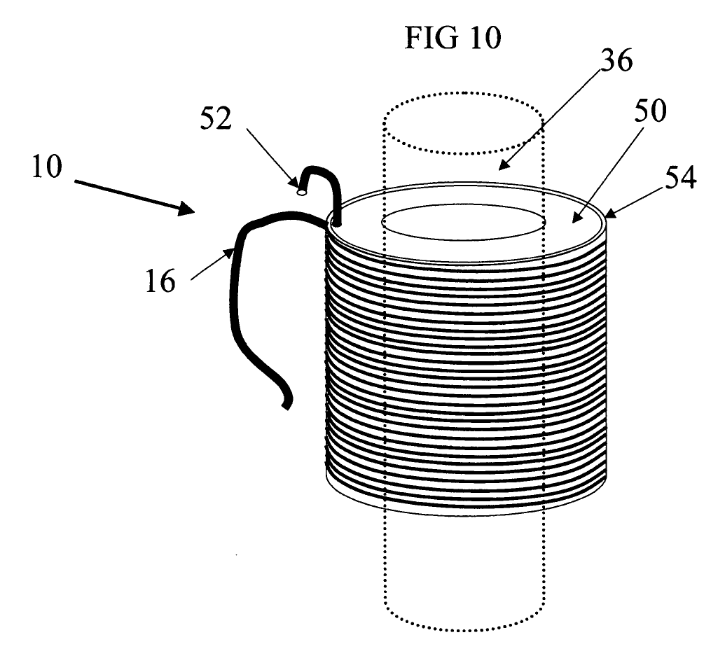

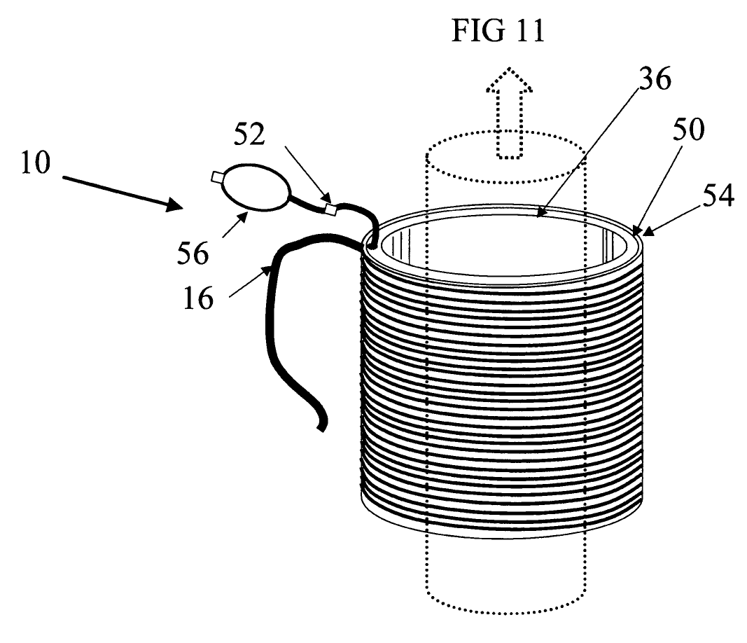

FIG. 9 illustrates the placement of a PES appliance 10 around a limb of a patient 36. When being placed, the self-inflating foam layer 50 is deflated. The opening in the center of the appliance 10 dilates allowing for the free admission of the patient's limb through the center of the solenoid. Once around the target limb, as illustrated in FIG. 10, the self-inflating foam layer 50 is allowed to inflate, securely holding the appliance 10 to the target limb 36.

As illustrated in FIG. 11 one embodiment of the present invention provides a pump 56 for the deflation of the self-inflating foam layer 50. One skilled in the art will readily appreciate that the pump may be either manual or automatic. In alternative embodiments where the layer 50 is not self-inflating, the pump may also be used to inflate the inflatable layer 50. When the layer 50 is deflated, the target limb may be readily removed from the appliance.

According to one embodiment a pulsed electrical signal is generated by the controller unit 22 and transmitted through the appliance 10. The electrical pulse then generates a magnetic signal or pulse in the appliance. This signal may be generated in a variety of different waveforms. In some instances this waveform is asymmetrical, in contrast to symmetrical waveforms such as sinusoidal or square waveforms. According to one such embodiment, the signal may be in a “peak and decay waveform” wherein the pulse is strongest at the beginning of a period and decays asymptotically toward zero prior to the beginning of the next period. The rise time of such an embodiment may be from between 0.1 ms (milliseconds) to 10 ms. The amplitude of the magnetic signal generated is between 5 and 200 Gauss. The waveform may have a single or narrow band frequency of between 5 and 60 Hz. According to one embodiment, a 30 Amp electrical pulse producing a 100 Gauss magnetic signal is provided. This pulse is produced in a 15 Hz peak and decay waveform, with a 0.5 ms rise time. Alternative embodiments wherein the electrical pulse is between 20-50 Amps would likewise be within the scope of the invention.

The use of single or narrow frequency pulsed electromagnetic fields reduces noise resulting from interfering frequencies. This is believed to improve the penetration of the signal and energy, and limits the signal to those frequencies believed by the clinician to be of therapeutic benefit.

A schematic of one embodiment of the present invention is illustrated in FIG. 14. This embodiment is composed of two parts: the coil 11 and the pulse generator 22. The coil 11 has the purpose of producing in the body part to which it is applied a magnetic field of suitable characteristic for the therapy to be effective, when the coil is driven by a current of suitable characteristics. The pulse generator 22 has the purpose of driving the coil 11 with the current.

The coil 11 of one embodiment of the present invention is a foldable coil, made of 80 turns of super flexible wire, of about 80 cm length for each turn, enclosed in a wrap of fabric. The wire has to be super flexible so that the whole coil can be wrapped around the body part where the magnetic field is needed.

The number of turns is determined by a reasonable compromise between the competing needs of producing a very large current to drive the coil 11, if the turns are too few, or producing a very large voltage to drive the coil 11, if the turns are too many. With 80 turns the magnetic field, with the coil tightly wrapped around a leg of normal size, is between 3.5 and 4 gauss for each A of current in the coil. This leads to a current of 50 A peak for a peak field of between 175 and 200 gauss. 50 A is quite a large current, but in short pulses, it is still quite manageable with normal electronic components. In other embodiments a less powerful current of 30 A or less may be used to induce a field of 110 Gauss in a similarly wrapped coil.

According to such an embodiment, the magnetic field rises from zero to the peak value in a time less than or equal to 0.5 ms, the current must rise in the same time, because the field waveform follows exactly the waveform of the current. The coil 11 has an inductance and an electrical resistance, both depending on its physical dimensions. For the coil 11 with rise time as described above the required voltage is about 150 V. The magnetic field thus produced by the coil 11 in such a configuration is parallel to the bone.

One skilled in the art will readily appreciate that other coil configurations may be employed having either more coils or fewer coils. For example, a wrap having a reduced weight may be obtained by decreasing the number of coils. An increase in current strength can compensate for the reduction in field strength resulting from the decreased coil count. According to one such embodiment, a 50% reduction in the number of coils is balanced with a doubling of the strength of the current. In such an embodiment, the peak intensity is unchanged, while the rise and decay times are halved.

The pulse generator of one embodiment as illustrated in FIG. 14 comprises: a DC power supply 62, of adequate output voltage, and capable of giving current pulses of the peak value, duration etc. as required by the powered circuit; The DC power supply 62 does not need to be stabilized; a controlled switch 66; means for measuring the instant current flowing in the switch, current transducer, or current sensor 68, a diode 64, a means for controlling the switch 66, turning it on at a predefined frequency, and turning it off as the current reaches a predefined value 70.

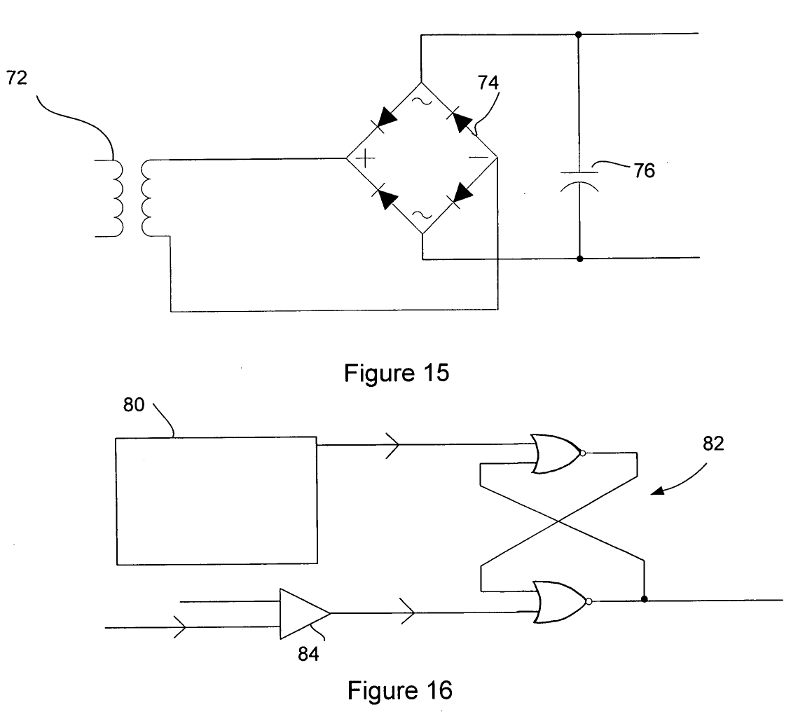

According to one embodiment, illustrated in FIG. 15, the power supply 62 is a DC power supply powered by the domestic 110 V AC power grid. It comprises an AC transformer 72, a bridge rectifier 74, and a filter capacitor 76. The capacitor 76 flattens the pulsed voltage emitted by the rectifier 74 and acts as a reservoir, such that when the switch is closed, the current comes primarily from the capacitor 76 rather than from the transformer 72. Such a configuration, among other benefits enables a more compact and lighter transformer 72 to be used.

As illustrated in FIG. 14, the power supply 62, switch 66, and current sensor 68 are connected to each other in series and series connected to the coil 11, such that when the switch 66 closes, the whole DC voltage produced by the power supply 62 is applied to the coil 11. The resistance of the switch when on, and the resistance of the current monitor, are low enough to be ignored. The diode 64 is connected to the coil 11 in parallel, with polarity such as not to conduct when the switch 66 closes and thereby applying voltage to the coil 11. In some embodiments, the DC power supply 62 does not need to be stabilized, as the value at which the current ceases to increase and starts to decrease is determined by the current transducer and the control circuit 70, as a result, even if the value of the DC voltage produced varies, the peak current value will not.

The control circuit 70, according to one embodiment illustrated in FIG. 16, comprises an oscillator 80, a latch or logic circuit 82 and a voltage comparator 84. The oscillator 80 emits signals at the same frequency as desired for the current pulses. These signals trigger the closing of the switch 66, and thereby initiate current pulses. The voltage comparator 84 emits a signal when the voltage coming from the current sensor 68 has reached a preset level corresponding to the desired peak current. The signal from the oscillator 80 sets the latch 82, which, when set, drives the switch in conduction. The signal from the comparator resets the latch, ending the conduction of the switch as the current has reached the preset value.

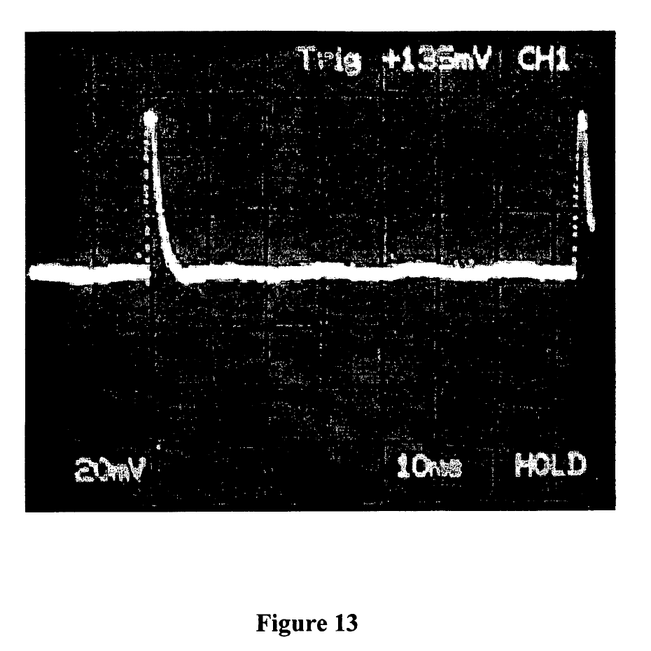

In this way, the control circuit 70 receives the current value measured by the current meter 68, and drives the switch 66 between the closed and open states. When the device is powered the circuit 70 periodically drives the switch 66 to the closed state, and keeps it closed until the current value measured by current sensor 68 reaches the predefined peak value. While the switch 66 is closed, the whole voltage V supplied by the power supply is applied to the coil, which has inductance L and resistance R. At first, the current starts rising with a slope equal to V/L, then the slope decreases as the voltage developed across R is subtracted from V and decreases the voltage applied to L. A photo of an oscilloscope graphing the waveform produce is illustrated in FIG. 13 wherein the frequency is 15 Hz and the peak intensity of the magnetic field produced is 500 Gauss.

The value of V was chosen accordingly to both R and L values, so as to give the wanted total rise time, less than 0.5 ms. As the current reaches the predefined peak value, the circuit drives the switch open. The current in the coil 11 cannot stop flowing, due to L. The voltage across the coil 11 changes and becomes slightly negative; this brings the diode 64 into conduction. The current continues to flow trough the diode 64, and the slope changes to downward, decaying exponentially because of the losses in R.

The whole process of the current rising to the peak value and falling again to the zero value take less time than the predefined time between two consecutive pulses, which is predefined in the circuit. So, when the predefined time has elapsed, and drives the switch to the closed state again, the current has already fallen to zero, and the process repeats itself in exactly the same way.

The current sensor 68 utilizes a shunt resistor 78 is provided. The shunt resistor 78 is a resistor of low enough resistance so as to not interfere with the operating of the circuit to which it is series connected. It is of known resistance and the voltage developed across it by the flowing current can be used as a measure of the current value. This information is then relayed to the control circuit 70.

According to one embodiment, a conductive layer 14 is connected to ground and is disposed between the coil 11 and the target member, a second current sensor 82 is disposed between the conductive layer 14 and ground. The second current sensor 82 is coupled to a switch 84 disposed such that when said switch 84 is open, the current to the pulse control and generation system 22 is interrupted. In this way the conductive layer 14, the sensor, 82 and the switch 84 act as a distributed ground fault interrupt. One skilled in the art will readily appreciate other and various circuits may be designed to achieve similar effects, and that such circuits would be within the scope of the present invention.

Some embodiments of the present invention include a variety of additional features. Among such additional features, a means may be provided for verifying that the pulse current are effectively flowing through the coil, and for lighting a LED on the front panel if they are flowing. This is mainly to ensure that the coil is connected and is not broken. Other alarms and indicators may be provided whereby the user may be alerted to problems within the device. In the event of either intentionally or as a result of accidental disconnection or interruption in the power supply of the device, the control circuit 70 temporarily suspends device operation, and upon reconnection or restart, the device will resume operations at the point in the cycle at which it was terminated. Various means for resetting the device may be provided including the depression of a combination of buttons or a dedicated reset button. Adjustable controls on the front panel may also be provided for selecting the frequency of the pulses and peak current value, though one skilled in the art will readily appreciate that in some embodiments the frequency and intensity of the pulses need not be adjustable.

Alternative embodiments may be susceptible to other variations. Two timers may be provided, either separate from or integrated into the control circuit 70, one whereby the user can control the duration of a therapeutic session and a second whereby usage is limited to the prescribed time per day. According to one embodiment, the memory required to preserve the timers is powered by a 9 volt battery or other power storage device during an interruption of the primary power supply to the device.

The first timer, or “user timer”, for the user to program the time duration of a therapy session. For instance, the user may have an input control to select the duration of the therapy session, and a start/stop pushbutton to start and temporarily stop the therapy. When the total time for which the device has been operating will reach the programmed time, the device will automatically stop. A user timer configured according to one embodiment may provide 6 preset time selections, ranging from 10 to 60 minutes in duration at 10-minute increments. Alternatively, similar indicator lights may be employed to indicate the time remaining in a selected treatment session. The selection may be changed during operation and takes immediate effect. In some embodiments, light emitting diode indicators may be lit to indicate the selected time.

According to one embodiment, a second timer, or “anti-abuse” timer, may be set by the factory, physician, clinician, or retailer to prevent the user from having the device running for more than 2 hours total in each 20-hour period. Other embodiments may be set to different time periods and limits based on testing indicating the efficacy and safety of various periods of exposure. According to one embodiment, the 20-hour time period is preserved in memory even in the event of a power failure to the device.

Results from clinical tests of one embodiment of the present invention and its efficacy in treating osteoarthritis knee pain are summarized in the following tables. Subjects pain was measured by primary end points including: modified WOMAC index for knee osteoarthritis, and 100 mm VAS as assessed both after 4 weeks of treatment sessions.

The test was a randomized, placebo controlled, double-blind trial to evaluate efficacy of the investigational device in reducing pain and improving function in persons with osteoarthritis of the knees.

Global WOMAC

| | Treatment Group % | Control Group % | |
|---|---|---|---|
| Week | Change from Baseline | Change from Baseline | P-value |
| 1 | −10% | 14% | 0.52 |
| 3 | 51% | 15% | 0.02 |
| 4 | 51% | 23% | 0.10 |

Functional Disability Scale—WOMAC

| | Treatment Group % | Control Group % | |
|---|---|---|---|
| Week | Change from Baseline | Change from Baseline | P-value |
| 1 | 32% | −16% | .078 |
| 3 | 53% | 17% | 0.12 |
| 4 | 55% | −1% | 0.06 |

Stiffness Scale—WOMAC

| | Treatment Group % | Control Group % | |
|---|---|---|---|
| Week | Change from Baselin | Change from Baseline | P-value |
| 1 | −11% | 20% | 0.43 |
| 3 | 2% | 16% | 0.76 |
| 4 | 68% | 28% | 0.04 |

Pain Scale—WOMAC

| | Treatment Group % | Control Group % | |
|---|---|---|---|
| Week | Change from Baseline | Change from Baseline | P-value |
| 1 | 8% | 3% | 0.70 |
| 3 | 14% | 10% | 0.90 |
| 4 | 7% | 3% | 0.88 |

Pain—VAS

| | Treatment Group % | Control Group % | |
|---|---|---|---|
| Week | Change from Baseline | Change from Baseline | P-value |
| 1 | 4% | −31% | 0.66 |
| 3 | 33% | −53% | 0.28 |
| 4 | 21% | −63% | 0.26 |

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A system for pulsed electromagnetic stimulation of a target member, the system comprising:
a pulse control and generation unit;
a conformable member appliance coupled to said pulse control and generation unit;
an insulated conductive material disposed within said appliance and proximate to said target member and producing a pulsed magnetic field when an electrical pulse having an asymmetric waveform is passed through said conductive material by said pulse control and generation unit.

2. The system of claim 1 wherein said conformable member comprises an inflatable layer.

3. The system according to claim 2 wherein said inflatable layer comprises a layer of self-inflatable foam.

4. The system according to claim 3 further comprising a pump coupled to said layer of self-inflating foam such that said self inflating foam can be deflated for insertion of said target member into said appliance.

5. The system according to claim 4 wherein said pump is selected from the group of pumps consisting of manual and automatic pumps.

6. The system according to claim 1 wherein said conductive material comprises at least one spring tensioned wire.

7. The system according to claim 6 wherein said appliance comprises an expandable sleeve.

8. The system according to claim 7 wherein said expandable sleeve comprises an expandable, conformable fabric selected from the group of fabrics consisting of man made stretch fabrics, knitted fabrics, elasticized fabrics, latex, and rubber.

9. The system according to claim 1 wherein said insulated conductive material is arrayed in a modified Helmholtz configuration such that first and second coils have an axis normal to an axis of said appliance, said coils conform to the shape of said target member and have a maximum separation equal to the diameter of said coil.

10. The system according to claim 1 wherein said insulated conductive material is configured in first and second coils arrayed in a classic Helmholtz configuration.

11. The system according to claim 1 wherein said insulated conductive material is substantially solenoidal in configuration, and comprises flexible wires configured for folding without damage.

12. The system according to claim 11 wherein said appliance is substantially larger than the radius of the target member and is configured to fold over and fasten.

13. The system according to claim 1 wherein said appliance further comprises a conductive layer connected to ground whereby said controller is triggered to interrupt said electrical pulse in the event of a failure in insulation of said conductive material.

14. The system according to claim 13, wherein said conductive layer is disposed between said insulated conductive material and at least one exterior insulating layer.

15. The system according to claim 1 wherein said electrical pulse has a current of between 20 and 50 amps.

16. The system according to claim 1 wherein said a pulse control and generation unit comprises:
a current sensor, monitoring electrical current flow through said insulated conductive material;
a control circuit, receiving data from said current sensor;
a switch controlled by said control circuit;
a diode disposed within said pulse generation and control circuit such that when said switch is open, said electric current flows through said insulated conductive material will decay.

17. The system according to claim 16, further comprising a resistor disposed in series with said insulated conductive material.

18. The system according to claim 16 wherein said control circuit comprises:
an oscillator, said oscillator controlling the frequency of said electrical pulse;
and a voltage comparator where by a reference voltage is compared to a signal produced by said current sensor.

19. The system according to claim 16 wherein said a pulse control and generation unit further comprises a first timer where by a user can program said system to provide said pulsed electromagnetic field for a desired time period.

20. The system according to claim 16 said pulse control and generation unit further comprises a second timer whereby said system may be programmed to provide said pulsed electromagnetic field for no longer than a prescribed time period.

21. A system for pulsed electromagnetic stimulation of a target member: said system comprising:
a pulse control and generation unit;
a conformable member appliance coupled to said pulse control and generation unit, said conformable member appliance comprising at least one coil of insulated conductive material and at least one grounded conductive layer disposed between said coil and said target member coupled to said pulse generation and control unit, such that an electrical current in said conductive layer at least temporarily disables said pulse control and generation unit;
said coil producing a pulsed magnetic field when a plurality of electrical pulses having asymmetric waveforms and peak currents between 20 A and 50 A is passed through said coil by said pulse control and generation unit.
